(19) United States
(12) Patent Application Publication
(10) Pub. No.: US 2015/0072966 A1
(43) Pub. Date: Mar. 12, 2015

(54) METHOD OF PROMOTING WOUND HEALING

(71) Applicant: 3M INNOVATIVE PROPERTIES COMPANY, SAINT PAUL, MN (US)

(72) Inventors: Joseph V. Boykin, JR., Richmond, VA (US); Stephanie F. Bernatchez, Woodbury, MN (US); Vinod P. Menon, Woodbury, MN (US); Joseph J. Stoffel, Hastings, MN (US); Joseph A. Tucker, Shoreview, MN (US)

(73) Assignee: 3M INNOVATIVE PROPERTIES COMPANY, SAINT PAUL, MN (US)

(21) Appl. No.: 14/381,636

(22) PCT Filed: Feb. 28, 2013
(86) PCT No.: PCT/US13/28127

§ 371 Date: Aug. 28, 2014

Related U.S. Application Data
(60) Provisional application No. 61/605,726, filed on Mar. 1, 2012.

(51) Int. Cl. G01N 33/84; A61K 31/198; A61K 31/573

(52) U.S. Cl. CPC G01N 33/84; A61K 31/573; A61K 31/198; G01N 2800/20
USPC 514/179; 514/565; 436/110

(57) ABSTRACT

A method of establishing a therapeutic window of wound fluid nitric oxide (WFNO) in the wound of a mammal, the method including: obtaining a wound fluid sample from a mammal; analyzing the WFNO level; determining whether the WFNO is at or below a lower threshold level, or is at or above an upper threshold level; wherein the lower threshold level and upper threshold level define the therapeutic window of WFNO; and treating the mammal with a substance that alters the WFNO level such that the therapeutic window of WFNO in the wound is established.

### BACKGROUND

NO is a small, hydrophobic gaseous free radical which is an important physiological mediator for autonomic functions such as vasodilatation, neurotransmission, and intestinal peristalsis. NO provides cellular signaling by activation of its target molecule, guanylate cyclase, which elevates intracellular concentrations of cyclic guanosine monophosphate (cGMP). Cellular signaling is performed without mediation of channels or cellular membrane receptors and is dependent upon the concentration of NO in the cellular environment.

Nitric oxide synthase (NOS) produces nitric oxide (NO) in the tissue. The NO has a half-life of 5 seconds in biological tissues. NO is normally metabolized to stable NO-related compounds (e.g., nitrate and nitrite compounds), which may be assayed in urine, plasma, tissue, wound fluid, or other specimens from a patient. The level of nitrate or nitrite compounds in a specimen can serve as an indicator of the level of NO synthesis in a patient.

### SUMMARY

The present disclosure is based on the discovery that below a lower threshold level of NO in the wound of a mammal and above an upper threshold level of NO in the wound of a mammal, normal wound repair is not achieved, resulting in a chronically nonhealing wound.

In one embodiment, a method is provided for promoting wound healing in a mammal. The method comprises treating the mammal with a substance that alters the wound fluid nitric oxide (WFNO) level such that of therapeutic window of WFNO in the wound is established thereby promoting wound healing in the mammal. The therapeutic window of WFNO is established by a method comprising obtaining a wound fluid sample from a mammal, analyzing the WFNO level, determining whether the WFNO is at or below a lower threshold level, or is at or above an upper threshold level. The lower threshold level and upper threshold level define the therapeutic window of WFNO.

In one embodiment, a method is provided for establishing a therapeutic window of wound fluid nitric oxide (WFNO) in the wound of a mammal. The method includes: obtaining a wound fluid sample from a mammal; analyzing the WFNO level; determining whether the WFNO is at or below a lower threshold level, or is at or above an upper threshold level; wherein the lower threshold level and upper threshold level define the therapeutic window of WFNO; and treating the mammal with a substance that alters the WFNO level such that the therapeutic window of WFNO in the wound is established.

In one embodiment, a method is provided for establishing a therapeutic window of wound fluid nitric oxide (WFNO) in the wound of a mammal. The method includes: obtaining a wound fluid sample from a mannnal; analyzing the WFNO level; wherein WFNO comprises a nitrate, a nitrite, or a combination thereof, in the wound fluid; determining whether the WFNO is at or below a lower threshold level, or is at or above an upper threshold level; wherein the lower threshold level and upper threshold level define the therapeutic window of WFNO; and treating the mammal with a substance that alters the WFNO level such that the therapeutic window of WFNO in the wound is established thereby allowing for improved wound healing.

In one embodiment, a method is provided for establishing a therapeutic window of wound fluid nitric oxide (WFNO) in the wound of a mammal. The method includes: obtaining a wound fluid sample from a mammal; wherein obtaining a wound fluid sample comprises: providing a sample acquisition device that is substantially free of reactive nitrates; contacting the sample acquisition device with a wound site for a period of time sufficient to collect wound fluid; and extracting a portion of the wound fluid from the sample acquisition device; analyzing the WFNO level; wherein WFNO comprises a nitrate, a nitrite, or a combination thereof, in the wound fluid; and further wherein analyzing the WFNO level comprises: providing a reducing agent capable of reducing a nitrate compound to a nitrite compound; providing a chromogenic reagent capable of reacting with a nitrite compound to form a colored compound (typically, a chromogenic reagent includes a diazotizing agent and a coupling agent); contacting the chromogenic reagent and reducing agent with the wound fluid sample under conditions that permit the reduction of a nitrate compound to a nitrite compound and permit the reaction of a nitrite compound with the chromogenic reagent to form the colored compound; and detecting the colored compound; determining whether the WFNO is at or below a lower threshold level, or is at or above an upper threshold level; wherein the lower threshold level and upper threshold level define the therapeutic window of WFNO; and treating the mammal with a substance that alters the WFNO level such that after said treating, the therapeutic window of WFNO in the wound is established thereby allowing for improved wound healing.

In one embodiment, a substance that alters the level of wound fluid nitric oxide (WFNO) for use in a method of treating a wound in a mammal or promoting wound healing in a mammal, the method comprising analyzing the WFNO level in a wound fluid sample obtained from the wound of the mammal; and administering said substance to the mammal in an amount to establish a WFNO level in the wound between a lower threshold level required for wound healing and an upper threshold level required for wound healing. If the WFNO level in said wound fluid sample is above the upper threshold level required for wound healing, the substance to be adminstered is one that decreases the WFNO level in the wound; or if the WFNO level in said wound fluid sample is below the lower threshold level required for wound healing, the substance to be administered is one that increases the WFNO level in the wound.

In one embodiment, an in vitro method of monitoring the effectiveness of the treatment of a wound in a mammal comprises analyzing the level of wound fluid nitric oxide (WFNO) in a wound fluid sample obtained from the wound of the mammal determining whether or not the WFNO level in the wound is between a lower threshold level and an upper threshold level required for wound healing.

Herein, “WFNO” refers to wound fluid nitric oxide (NO) and NO-related compounds (e.g., one or more metabolites of NO), and combinations thereof, contained in wound fluid. NO-related compounds include nitrate compounds and nitrite compounds, collectively referred to herein as “NOx.”

The terms “comprises” and variations thereof do not have a limiting meaning where these terms appear in the description and claims.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or all of the listed elements or a combination of any two or more of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The present invention will be further explained with reference to the drawing figures listed below, where like structure is referenced by like numerals throughout the several views.

FIG. 1a is a side view of one embodiment of a sample acquisition device according to the present disclosure.

FIG. 1b is a top view, partially in section of the sample acquisition device of FIG. 1a.

FIG. 2 is a graph of wound fluid NOx levels from venous leg ulcer (VLU) patients.

FIG. 3 is a graph of wound fluid NOx levels from diabetic foot ulcer (DFU) patients.

### DETAILED DESCRIPTION OF ILLUSTRATIVE EMBODIMENTS

The wound may be a result of trauma (e.g., a puncture wound), a medical condition (e.g., a pressure ulcer or an abscess), or a surgical wound (e.g., an incision), to skin tissue or a mucous membrane. The present disclosure is based on the discovery that at or below a lower threshold level of NO in the wound of a mammal and at or above an upper threshold level of NO in the wound of a mammal, normal wound repair is typically not achieved, resulting in a chronically nonhealing wound.

In one embodiment, a method is provided for establishing a therapeutic window of wound fluid nitric oxide (WFNO) in the wound of a mammal. The method includes: obtaining a wound fluid sample from a mammal (preferably, a human); analyzing the WFNO level; determining whether the WFNO is at or below a lower threshold level, or is at or above an upper threshold level; wherein the lower threshold level and upper threshold level define the therapeutic window of WFNO; and treating the mammal with a substance that alters the WFNO level such that the therapeutic window of WFNO in the wound is established.

If the level of WFNO is outside this therapeutic window (i.e., at or above the upper threshold level, or at or below the lower threshold level), the wound is typically considered a chronic wound, a non-progressive wound, or a chronically nonhealing wound. For example, for wounds with a low WFNO level (i.e., those at or below the lower threshold level), the wounds are generally non-progressive wounds with worsening wound status. For wounds with a high WFNO level (i.e., those at or above the upper threshold level), the wounds are generally non-progressive wounds with excessive wound inflammation and wound bed stagnation. As used herein, these terms refer to a wound that does not heal in a normal time frame of healing compared to a subject of similar age and health condition. Typically, a wound is chronic if it has not healed in months or years and can be characterized by one or more of the following: necrotic tissue, purulent exudate, excessive exudate, or offensive odor.

If the therapeutic window of WFNO in the wound is established (i.e., the level of WFNO is within this therapeutic window such that it is below the upper threshold level and above the lower threshold level), then the wound is typically considered a “normal wound” or a “non-chronic wound” or a wound of a “normal subject.” Such wounds generally heal in a normal time frame (e.g., days or weeks) or show progressive wound improvement with healing or successful closure (e.g., skin grafting).

It will be understood that there are always exceptions to this general rule regarding the level of WFNO in a wound. For example, it will be understood that some wounds may be non-healing due to other reasons, such as excessive bacterial load. However, the identification of a therapeutic window of WFNO provides significant guidance to a practitioner in determining a therapeutic procedure for enhancing wound healing.

Herein, “WFNO” refers to wound fluid nitric oxide (NO) and NO-related compounds (e.g., one or more metabolites of NO), and combinations thereof, contained in wound fluid. NO-related compounds include nitrate compounds and nitrite compounds. Nitrate and nitrite compounds are collectively referred to herein as “NOx.” Wound fluid NOx levels (nitrate and nitrite levels) are referred to herein as WFNOx.

In addition to nitrite and nitrate, other molecular species related to NO synthesis or breakdown (other “NO-related compounds”) can be quantified in wound fluid. For example, levels of L-citrulline, which is a product of the reaction that produces NO, or cGMP, which is produced as a result of NO activation of guanylate cyclase, can be determined as a reflection of systemic NO synthesis in a patient (F. L. Kiechle et al., Ann. Clin. Lab. Sci., 26(6), 501 (1996)). Similarly, L-dimethylarginine, another product of NOS, can be detected by HPLC and used as a highly specific index of systemic NOS activity (J. Meyer et al., Anal. Biochem., 247(1), 11 (1997)). NO can also break down by reacting with superoxide anion in human plasma to produce peroxynitrite, which in turn can produce a variety of radicals such as ascorbyl radical and albumin-tinyl radical that can be detected using electron paramagnetic resonance (EPR) spectroscopy (L. Vasquez-Vivar et al., Biochem. J., 314, 869 (1996)). Another product of peroxynitrite is 3-nitrotyrosine, which can be detected by gas chromatography in tandem with mass spectrometry (E. Schwedhelm et al., Anal. Biochem., 276(2), 195 (1999)), reversed-phase HPLC (H. Ohshima et al., Nitric Oxide 3, 132 (1999)), or an ELISA method using anti-nitro tyrosine antibodies (J. C. ter Steege et al., Free Radic. Biol. Med., 25(8), 953 (1998)). Furthermore, the in situ detection of NO itself is possible with the aid of biosensors that quantify NO levels and changes in NO levels in response to stimuli. For example, the heme domain of soluble guanylate cyclase, a natural receptor for NO, can be labeled with a fluorescent reporter dye, and changes in fluorescence intensity can be determined through an optical fiber and calibrated to reveal NO levels at any desired location in the body, for example at or near a wound site (S. L. Barker et al., Anal. Chem., 71, 2071 (1999)). Other NO-related compounds that can be measured include cGMP (cyclic guanosine monophosphate), N-nitroso groups such as S-nitroso-L-cysteine and S-Nitroso-L-glutathione (GSNO) and chemically synthesized S-nitrosothiols. Also included are those obtained by metabolic nitrosation of suitable substrates such as proteins and amino acids mediated by nitrite, as well as 5-nitroso adducts, metal-NO complexes, nitosoamines and nitrosoamides.

Given the rapid decomposition of NO in biological fluids, direct detection of NO should be performed in-situ rather than some time following collection of a specimen.

For NOx (i.e., the level of nitrate and/or nitrite compounds) in wound fluid, the lower threshold level is no greater than 10 micromolar, or no greater than 15 micromolar for a higher confidence level, and the upper threshold level is no less than 60, or no less than 50 micromolar for a higher confidence level. Thus, the therapeutic window for NOx can be within 10 to 60 micromolar, within 10 to 50 micromolar, within 15 to 60 micromolar, or within 15 to 50 micromolar. If the level of NOx is outside this therapeutic window (i.e., at or above the upper threshold level or at or below the lower threshold level), the wound is typically considered a chronic wound or a chronically nonhealing wound or a non-progressive wound (again, there are exceptions such as if the wound includes a high bacterial load).

If the level of NOx is within this therapeutic window such that it is below the upper threshold level (below 60 micromolar, or for a higher confidence level below 50 micromolar) and above the lower threshold level (above 10 micromolar, or for a higher confidence level, above 15 micromolar), then the wound is considered a normal wound or a non-chronic wound, thereby generally healing in a normal time frame (e.g., days or weeks) or showing progressive wound improvement with healing or successful closure (e.g., skin grafting) (again, there are exceptions such as if the wound includes a high bacterial load).

The wound fluid of a subject (i.e., mammal) is evaluated for the WFNO level (preferably, for the NOx level), and the wound is determined to be a chronic wound or a chronically nonhealing wound, methods of the present invention include treating the mammal with a substance that alters the WFNO level (preferably, the WFNOx level) such that the therapeutic window of WFNO in the wound is established. Herein, “establishing the therapeutic window of WFNO” in the wound (i.e., the level of WFNO is within this therapeutic window such that it is below the upper threshold level and above the lower threshold level) allows for improved wound healing. In this context, “improved wound healing” includes improved responsiveness to a wound healing therapy. Methods of the present disclosure can also involve monitoring the effectiveness of treatment.

If the WFNO is at or below the lower threshold, treatment can involve administration of a therapeutic agent or other treatment protocol designed to raise the level of nitric oxide in the subject. Examples of such therapeutic treatment protocols include administration of an effective amount of L-arginine to the mammal, subjecting the mammal to a hyperbaric oxygen treatment, treating the wound with topical nitric oxide in a gaseous form (as described in Anatoly B. Shekhter et al., Nitric Oxide, 12, 210-219 (2005), applying a nitric oxide donor (e.g., a polymer) to a wound (as described, for example, in U.S. Pat. Nos. 6,855,366 and 7,052,711, U.S. Pat. Appln. No. 2010/0098733, International Pub. Nos. WO 2006/058318, WO 2006/095193, WO 2009/155690, and WO 2005/003032, EP1690558, as well as in Yan Li et al., Molecular Pharmaceuticals, 7(1), 254-266 (2010), Maria Francisca Garcia-Saura et al., Journal of Investigative Dermatology, 130, 608-611 (2010), A. A. Eroy-Reveles et al., Future Med. Chem, 1(8), 1497-1507 (2009), and S. Y. Silva et al., Trials, 8:26 (2007)). Other methods of directly enhancing NO in the wound include the application of nanocrystalline NO powders or nitroglycerin paste. There are also some reported therapies that used specially filtered light sources to “release” NO from hemoglobin and nitrosothiols to increase intravascular NO to enhance wound healing. In addition, extremely low frequency electromagnetic fields have been shown to induce iNOS and eNOS, two enzymes leading to the production of nitric oxide (A. Patruno et al., British Journal of Dermatology, 162, 258-266 (2010)).

Gene therapy can also be used as this would include the use of iNOS or eNOS probes to increase endogenous NO production by increasing enzyme activity (e.g., as described in Jian-Dong Luo et al., Circulation, 110, 2484-2493 (2004) and Kokushi Yamasaki et al., and J. Clin. Invest., 101(5), 967-971 (1998)).

If the WFNO is at or above the upper threshold, treatment can involve administration of a therapeutic agent or other treatment protocol designed to decrease the level of nitric oxide in the subject. Examples of such therapeutic treatment protocols include the administration of an effective amount of a nitric oxide inhibitor (e.g., as described in U.S. Pat. No. 6,713,079), administration of an effective amount of one or more inhibitors of nitric oxide synthase (e.g., as described in M. R. Schaffer et al., European Journal of Surgery, 165(3), 262-7 (1999), administration of herbs with nitric oxide scavenging activity (e.g., as described in Ganesh Chandra Jagetia et al., Phytother. Res. 18, 561-565 (2004)), administration of steroids or topical application of corticosteroid preparations including cortisone, hydrocortisone (cortisol), prednisone, prednisolone, methyl-prednisolone, triamcinolone, dexamethasone and betamethasone (e.g., as described in A. Ahluwalia, Mediators of Inflammation, 7, 183-193 (1998)), glucocorticoids (e.g., as described in M. W. Radomski et al., Proc. Nati. Acad. Sci. USA, 87, pp. 10043-10047(1990)).

Also, treatment protocols can involve the use of nutritional supplements (e.g., protein-calorie malnutrition should be corrected with high protein/calorie supplements). Arginine supplementation may be provided to boost endogenous NO production. This can involve the administration of ARGINAID, a supplement made by Nestle, which includes arginine combined with high doses of vitamin C and E (antioxidants) without glucose (for use with diabetes), or gelatin (basically processed bone marrow) taken as gelatin drink or otherwise.

If the subject is currently on steroids for arthritis or chemotherapy, a treatment protocol could involve the reduction or discontinued use of steroids. Subjects could also be evaluated for risk factors that will lower or scavenge endogenous NO. This would include checking homocysteine values and treating elevated levels with high dose vitamin B6, B12 and folic acid. The cessation of cigarette smoking will also decrease oxidative stress and theoretically should enhance NO levels. Avoidance of high fat/cholesterol diets and use of approved antioxidants will also potentially enhance NO production.

Various combinations of these treatment protocols can be used if desired.

Following one or more treatment protocols, the subject can be monitored for effectiveness of the treatment by the method of obtaining a wound fluid sample, analyzing the WFNO level, and determining whether the WFNO is at or below a lower threshold level, or is at or above an upper threshold level, as described above. If such analysis determines that the WFNO is still outside the therapeutic window, then the effectiveness of the therapeutic treatment protocol is insufficient to promote wound healing. In that case, the treatment can be subsequently adjusted, for example by increasing the dose or potency of the therapeutic agent or increasing the period of exposure to the therapeutic agent. In a related embodiment, the method of monitoring the patient is repeated, and the dose or potency of the therapeutic agent, or period of exposure to the therapeutic agent, is again increased. Preferably, in this embodiment the method of monitoring and increasing the dose of the therapeutic agent is increased until the WFNO in a specimen from the subject is within the therapeutic window. It may be desirable to then maintain the therapy at the most effective dose as long as needed until the wounds of the patient have healed.

### Method of Collecting and Analyzing a Wound Fluid

Methods for detecting WFNO (wound fluid NO) in a wound typically involve collecting a sample of wound fluid from the wound site. Methods of the present disclosure can include the collection of wound fluid using a sample acquisition device, such as described below.

### Detection Methods:

Methods of the present disclosure include detecting endogenous NOx in the wound fluid. The NOx can be detected by methods that are known in the art, including those described herein.

NO is normally metabolized to certain stable products such as nitrate and nitrite. The level of nitrate, nitrite, or other NO-related products in a specimen serves as an indicator of the level of NO synthesis in a patient. NOx can be detected in a patient sample by methods that are known in the art, including, for example, spectrometry methods (e.g., colorimetric methods, fluorometric methods, and GC/mass spectrometry).

Methods of detecting NOx can be found, for example, in U.S. Patent Application Publication No. US 2003/0134332 and in an article by D. Tsikas (Anal. Chem., 72, 4064-4072 (2000)). The level of nitrate or nitrite in the specimen can be quantified by any method known in the art which provides adequate sensitivity and reproducibility. For example, the Griess reaction is a spectrophotometric assay for nitrate that can provide sensitive determination of nitrate and nitrite in biological fluid samples (M. Marzinzig et al., Nitric Oxide, 1, 177 (1997)). If the Griess reaction or another nitrite assay is performed both with and without reduction of nitrate to nitrite, then nitrate values can be obtained as the difference between the nitrite values obtained for the reduced sample and the non-reduced sample. The Griess assay can be made more sensitive if a fluorescent product is obtained, e.g., by reacting nitrite with 2,3-diaminonaphalene (T. P. Misko et al., Anal. Biochem., 214, 11 (1993)). Highly sensitive assays are also available which first reduce nitrite and nitrate (R. S. Braman and S. A. Hendrix, Anal. Chem., 61, 2715 (1989)) or any NO-related compound (M. Sonoda et al., Anal. Biochem., 247, 417 (1997)) to NO for detection with specific chemiluminescence reagents. A variety of protocols have also been described for detecting and quantifying nitrite and nitrate levels in biological fluids by ion chromatography (e.g., S. A. Everett et al., J. Chromatogr., 706, 437 (1995); J. M. Monaghan et al., J. Chromatogr., 770, 143 (1997)), high-performance liquid chromatography (e.g., M. Kelm et al., Cardiovasc. Res., 41, 765 (1999)), and capillary electrophoresis (M. A. Friedberg et al., J. Chromatogr. 781, 491 (1997)). Alternatively, the level of NO can be detected using the Sievers method (A. J. Dunham et al., Anal. Chem., 67, 220-224 (1995)), which is the method most commonly used to measure NO. In this method, the conversion of nitrite from biological fluids into NO for measurement in the gaseous phase is described.

The “level” of WFNO, and preferably NOx, refers to the concentration (in moles per liter, micromoles per liter, or other suitable units) of the respective product in the specimen, or in the fluid portion of the specimen. However, other units of measure can also be used to express the level of the products. For example, an absolute amount (in micrograms, milligrams, nanomoles, micromoles, moles, or other suitable units) can be used, particularly if the amount refers back to a constant amount, mass, or volume of patient specimen (e.g., grams, kilograms, milliliters, liters, or other suitable units). A number of commercially available kits can be used. For example, Cayman Chemical Company (Ann Arbor, Mich.) provides kits to detect nitric oxide metabolites colorimetrically or fluorometrically.

In some embodiments, NOx in a sample can be detected by the methods described in International Publication Nos. WO 2011/017317 and WO 2011/017325.

Nitric oxide has a very short half-life in vivo, so in some embodiments, the sum of the final metabolites nitrite (NO₂⁻) and nitrate (NO₃⁻) are measured. The presence of proteins in biological samples interferes with the colorimetric detection of this assay due to light scattering. In addition, hemoglobin (present if the sample is contaminated with blood) absorbs at the same wavelength as the chromophore formed in the reaction. Protein removal is therefore typically preferred prior to running the reaction and is accomplished by precipitation with an agent such as zinc sulfate, or by ultrafiltration through a porous membrane with a 10 kDa molecular weight cut-off (such as Millipore, Catalog No. UFC501096 or UFC801008), or by boiling and centrifuging or diluting the sample (http://www.oxfordbiomed.com/sites/default/files/spec_sheet/NB98.pdf).

Generally, a reducing agent is used to convert nitrate into nitrite. The nitrite concurrently reacts with one or more chromogenic reagents (e.g., a diazotizing agent and a coupling agent) to produce a red cationic dye which can be detected using a spectrophotometer.

Exemplary reducing agents include vanadium (III) chloride, or a cadmium/copper reagent, or reduction can also be achieved enzymatically using nitrate reductase, for example.

Exemplary diazotizing reagents include p-diaminodiphenyl sulfone, 4,4′-bis-(dimethylamino)thiobenzophenone, p-phenylazoaniline, p-nitroaniline, anthranilic acid, p-aminoacetophenone, p-aminophenylsulphone, p-phenylaniline, sulphanilic acid, bis-(4-aminophenyl)sulphide, (4-aminophenyl)trimethylammonium chloride, chloro-p-phenylenediamine, resorcinol, N,N-dimethylaniline, p-aminoacetophenone, 4-nitro-1-naphthylamine, p-nitroaniline, 4-nitro-1-naphthylamine, p-phenylazoaniline, p-nitroaniline, 4-nitro-naphthylamine, p-aminoacetophenone, 1-anilinonaphthalene. Of these, p-diaminodiphenyl sulfone (i.e., 4,4′-sulfonyldianiline) is particularly preferred.

Exemplary coupling agents include an aromatic diamine such as N-(1-naphthyl)-ethylenediamine dihydrochloride (NEDD), N-(2-diethylaminoethyl)-1-naphthylamine oxalate (Tsuda's reagent), N,N-dimethyl-1-naphthylamine, chromotropic acid, 1-naphthylamine, 1-naphthol, benzaldehyde 2-benzothiazolylhydrazone, anthrone, 1-anthrol, azulene, diphenylamine, 1,2-dihydroxybenzene, and sesamol. Of these, Tsuda's reagent is particularly preferred due to its oxidative stability.

In one embodiment, a method includes forming a mixture that includes a sample suspected of containing NOx and N-(1-naphthyl)-ethylenediamine. In one embodiment, the method of detecting NOx includes forming a mixture including a sample suspected of containing NOx and 4,4′-sulfonyldianiline. In one embodiment, the method of detecting NOx includes forming a mixture including a sample suspected of containing NOx, VCl₃, and HCl. In one embodiment, the method of detecting NOx includes forming a mixture including a sample suspected of containing NOx, VCl₃, HCl, and 4,4′-sulfonyldianiline. In one embodiment, the method of detecting NOx includes forming a mixture including a sample suspected of containing NOx, N-(1-naphthyl)-ethylenediamine, and 4,4′-sulfonyldianiline. In one embodiment, the method of detecting NOx includes forming a mixture including a sample suspected of containing NOx, VCl₃, HCl, and N-(1-naphthyl)-ethylenediamine. In one embodiment, the method of detecting NOx includes forming a mixture including a sample suspected of containing NOx, VCl₃, HCl, 4,4′-sulfonyldianiline, and N-(1-naphthyl)-ethylenediamine. Without being bound by theory, Reaction Scheme I shows a proposed pathway for the formation of a red cationic dye to detect nitrate in a mixture including a nitrate (NO₃), VCl₃, HCl, 4,4′-sulfonyldianiline, and N-(1-naphthyl)-ethylenediamine:

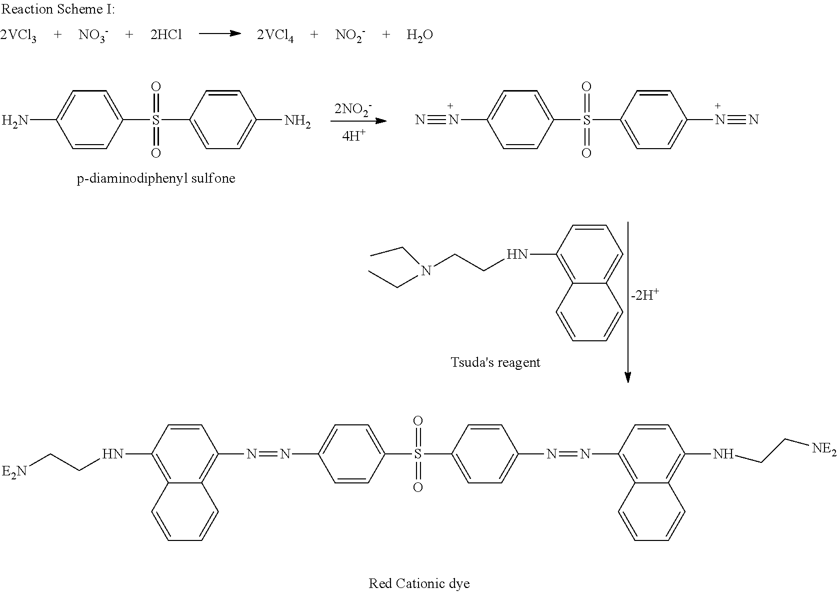

Reacting a sample suspected of containing NOx in a mixture including VCl₃, HCl, 4,4′-sulfonyldianiline and N-(1-naphthyl)-ethylenediamine can include reacting the mixture at an elevated temperature. Elevated temperatures can be used to increase the rate of the reaction, provided the elevated temperature does not substantially decrease the accuracy, sensitivity, and/or reproducibility of the reaction. For example, a temperature of 70° C. can be used. The reaction is typically quantitative in a temperature range of 25° C. to 100° C.

In one embodiment, a sample suspected of containing NOx can be reacted in a mixture including VCl₃, HCl, 4,4′-sulfonyldianiline and N-(1-naphthyl)-ethylenediamine for a period of time (e.g., 5 minutes to 24 hours) sufficient to form a detectable amount of red cationic dye. In a preferred embodiment, a sample suspected of containing NOx can be reacted in a mixture comprising VCl₃, HCl, 4,4′-sulfonyldianiline, and N-(1-naphthyl)-ethylenediamine at 70° C. for 10 minutes (or at 100° C. for 5 minutes or at 25° C. for up to 24 hours). In this embodiment, the method can be used to visually detect at least 50 pmoles of NOx in a 10 microliter (μL) sample by making a spectrophotometric reading of the resulting solution.

In any of the above embodiments, the method can further include cooling the reaction mixture. The reaction mixture can be cooled to room temperature, for example. In any of the above embodiments, the method can further include diluting the reaction mixture. The reaction mixture can be diluted with water (e.g., deionized water), for example. In some embodiments (e.g., those in which a retention medium is used in a filtration method), a reaction mixture of 170 μL can be diluted with 830 μL of deionized water.

In some embodiments, the method further can include filtering the mixture. The mixture can be filtered through any filtration media that is suitable to retain the red cationic dye that is a product of the reaction and that does not substantially interfere with the detection or quantitation of the red cationic dye. In some embodiments, the red cationic dye retained by the filter can be observed visually. In some embodiments, the red cationic dye retained by the filter can be detected or quantitated using an instrument (e.g., a reflection densitometer RD917, available from GretagMacbeth, Munich, DE). The reflection densitometer can be used with any suitable filter to detect a red-colored compound. In some embodiments, a green filter can be used to detect a red-colored compound.

Methods of the present invention can measure low levels (e.g., as low as 1-5 micromolar (μM)) of NOx in wound fluid with as little as 10 μL of sample. This assay is relatively fast (e.g., less than 45 minutes).

### Devices for Collecting Wound Fluid and Use

The methods of the present disclosure can use a wide variety of devices and methods for collecting a sample of wound fluid. Exemplary such devices are described in International Publication Nos. WO 2011/017325 and WO 2011/01731.

Preferred sample acquisition devices are substantially free of reactive nitrates and nitrites, which can interfere with the measurements of endogenous NOx in a wound. Using such devices, a sample can be collected rapidly and the NOx can be measured without interference from the sample acquisition device.

Devices for collecting a sample of wound fluid releasably acquire (e.g., by adsorption and/or absorption) an amount of wound fluid sufficient to perform one or more test procedures. In some embodiments, the device will releasably acquire at least 10 μL of wound fluid. In some embodiments, the device will releasably acquire at least 50 μL of wound fluid. In some embodiments, the device will releasably acquire at least 100 μL of wound fluid. In some embodiments, the device will releasably acquire at least 200 μL of wound fluid.

In some methods of use, it may be desirable to collect a sample of wound fluid and store it in the sample acquisition device for a period of time. Therefore, in some embodiments, it may be desirable to use a sample-collection device that is stable (e.g., maintains its structural and/or chemical stability) at the conditions (e.g., time, temperature, humidity, etc.) in which the device will be stored.

In some methods of use, it may be desirable to store the wound fluid in the sample-collection device before the wound fluid is removed from the sample acquisition device. For example, it may be desirable to freeze a device containing a sample, in order to preserve the sample for subsequent testing. Therefore, in some embodiments, it may be desirable to use a sample acquisition device that is stable to the process (e.g., freezing). An example of a suitable sample acquisition device is a NEXCARE Soft 'n Flex first aid dressing (catalog no. 672-35), available from 3M Company (St. Paul, Minn.). The 3M Nexcare “Soft 'n Flex” First Aid Dressing (FAD) absorbent pad is a polyethylene/polyester/EVA polymer with a surfactant treatment and TiO2. The pads are STRATEX material from DelStar Technologies Inc., of Middletown, Del. The NEXCARE first aid dressing can collect up to several hundred microliters of wound fluid, can be processed (e.g., by freezing) and, does not substantially interfere with a plurality of tests for analytes that are endogenous to a wound site.

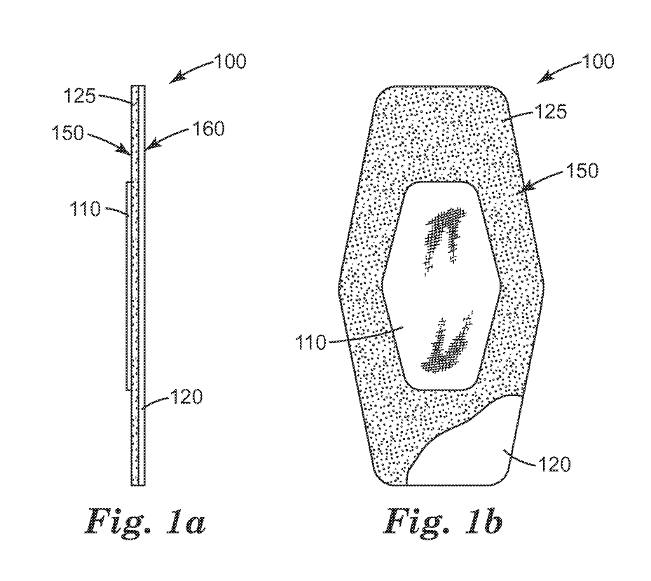

FIG. 1a shows a side view of one embodiment of a sample acquisition device 100 according to the present disclosure. The device 100 comprises an absorbent pad 110, which may be coupled to an optional backing 120. The backing 120 may further comprise an adhesive layer 125, which can function to couple the absorbent pad 110 to the backing 120.

The absorbent pad 110 comprises at least one absorbent material capable of adsorbing and/or absorbing wound exudate comprising fluid. The absorbent material can be a fibrous material (e.g., polymeric fibers) or a foam material (e.g., an open-cell foam or a closed-cell foam). The absorbent material is substantially free of components that may interfere with a test procedure to detect NOx in a wound exudate. An example of a preferred absorbent fiber is a polyester fiber (e.g., DACRON polyester fibers). In some embodiments, the polyester fibers may comprise a coating (e.g., a hydrophilic coating). In some embodiments, the absorbent pad 110 may further comprise an outer layer (not shown). The outer layer can function to facilitate the movement of wound exudate from the wound site to the absorbent material and/or to retain the absorbent material.

The backing 120 may be constructed from a number of suitable materials including, for example, metal (e.g., a metal foil), glass, a film (e.g., a plastic film), and combinations thereof. In some embodiments, the backing 120 is substantially water resistant. In some embodiments, the backing 120 may comprise pores and/or perforations (not shown), which permit the passage of gas (e.g., air) and/or liquids through the backing. In certain embodiments, it may be desirable to decontaminate, disinfect, or sterilize the device 100 before use. In these embodiments, the materials for the absorbent pad 110 and/or the backing 120 can be selected for their compatibility with the decontamination, disinfection, or sterilization process.

FIG. 1a shows that the device 100 comprises two major surfaces: first major surface 150 and second major surface 160, respectively. In this embodiment, the first major surface 150 includes the absorbent pad 120 and, thus, it is the side of the device 100 that can be oriented toward a wound site (not shown) to allow direct contact between the absorbent pad 110 and the wound. In some embodiments (e.g., in an embodiment where the backing 120 comprises perforations (not shown)), the second major surface 160 of the sample acquisition device 100 can be contacted with the wound site and the wound exudate can pass through the perforations and onto and/or into the absorbent pad.

FIG. 1b shows a top perspective view, partially in section, of the sample acquisition device 100 of FIG. 1a. It can be seen that the backing 120 extends outside the perimeter of the absorbent pad 110. In use, when the absorbent pad 110 is contacted with a wound site, the adhesive layer 125 can be contacted with the area (e.g., skin) surrounding the wound site and the adhesive layer 125 can hold the sample acquisition device securely in place while the wound exudate is collected from the wound site.

In some embodiments, the wound fluid is collected by contacting the absorbent pad of a sample acquisition device directly against the wound site. In some embodiments, prior contacting the absorbent pad to the wound site, a dressing is removed from the wound. In some embodiments, the wound fluid is collected by contacting the absorbent pad indirectly with the wound site (e.g., the sample acquisition device is contacted with a drainage tube, a wound fluid collection container (e.g., a negative-pressure wound therapy device), or a wound dressing saturated with wound fluid).

During contact with the wound site, at least a portion of the wound fluid, which may contain some cells and/or fragments of cells, is transferred to the absorbent pad of the sample acquisition device. At least a portion of the wound fluid is retained (e.g., by absorption and/or adsorption) on and/or in the absorbent pad after the sample acquisition device is removed from contact with the wound site. The absorbent pad of the sample acquisition device is contacted with the wound for a period of time sufficient to collect a sample of wound fluid. In some embodiments, the absorbent pad of the dressing is contacted with the wound for 24 hours or less. In some embodiments, the absorbent pad is contacted with the wound for 30 minutes or less. In some embodiments, the absorbent pad is contacted with the wound for 10 minutes or less. In some embodiments, the absorbent pad is contacted with the wound for 5 minutes or less. In some embodiments, the absorbent pad is contacted with the wound for 1 minute or less. In some embodiments, the absorbent pad is contacted with the wound for 30 seconds or less. In some embodiments, the absorbent pad is contacted with the wound for 10 seconds or less.

Typically, the absorbent pad of the sample acquisition device is contacted with the wound site using manual pressure against the opposite side of the absorbent pad. The dressing is held in contact with the wound site until a sufficient amount of wound fluid is collected on and/or in the absorbent pad and the sample acquisition device is subsequently removed from contact with the wound. In some embodiments, the adhesive backing of the sample acquisition device can be folded back on itself, forming a convenient handle for a person to grasp the sample acquisition device while contacting it with the wound site. In an alternative embodiment, the absorbent pad of the first aid dressing is contacted with the wound while the adhesive backing is contacted with a surface (e.g., skin) adjacent the wound site. Advantageously, the adhesive backing holds the dressing in place for a period of time sufficient to collect a sample of wound fluid.

The absorbent pad may be repositioned one or more times at the wound site to collect additional wound fluid. The amount of wound fluid sufficient for analysis can depend on the test procedures used in the analysis. Typically, each analytical test may require 10 microliters to 100 hundred microliters of wound fluid.

Methods of collecting and analyzing wound fluid typically further include cleansing and/or irrigating the wound site, for example, with sterile water or a sterile solution that includes saline. The wound site can be cleansed before and/or after obtaining a sample with a sample acquisition device. In some embodiments, a first sample is obtained from a wound site with a first sample acquisition device, the wound is cleansed, a second sample is obtained with a second sample acquisition device described herein, the samples are extracted from the first and second sample acquisition devices, and the wound fluid from the sample acquisition devices is analyzed to detect NOx.

Methods of collecting and analyzing wound fluid typically further include processing the sample acquisition device. Processing the sample acquisition device includes, for example, processing the device for preserving, storing and/or transporting a sample of wound fluid. Non-limiting examples of processing a sample acquisition device include adding a reagent to the sample acquisition device and freezing (e.g., at −80° C.) a sample-laden sample acquisition device.

Methods of collecting and analyzing wound fluid typically further include extracting a portion of the wound fluid from the sample acquisition device. Extracting is used in the broadest sense of recovering at least a portion of the wound material from the sample acquisition device. In some embodiments, the wound material can be extracted by physical means (e.g., using pressure to express fluid from the sample acquisition device, using centrifugal force to separate wound fluid from the sample acquisition device, using an electromagnetic field to separate a portion of the wound fluid from the sample acquisition device), by chemical means (i.e., using a solvent, such as water or a buffer, for example, to extract the wound material from the sample acquisition device), or a combination of physical and chemical means to extract a portion of the wound fluid.

In certain preferred embodiments, the sample acquisition device is selected to provide highly-efficient extraction of the wound fluid. In some embodiments, the sample acquisition device may provide for extraction and recovery of at least 50% of the wound fluid. In some embodiments, the sample acquisition device may provide for extraction and recovery of at least 60% of the wound fluid. In some embodiments, the sample acquisition device may provide for extraction and recovery of at least 70% of the wound fluid. In some embodiments, the sample acquisition device may provide for extraction and recovery of at least 80% of the wound fluid. In some embodiments, the sample acquisition device may provide for extraction and recovery of at least 90% of the wound fluid. In some embodiments, the sample acquisition device may provide for extraction and recovery of at least 95% of the wound fluid.

### EXAMPLES

Objects and advantages of this disclosure are further illustrated by the following examples, but the particular materials and amounts thereof recited in these examples, as well as other conditions and details, should not be construed to unduly limit this disclosure.

Unless otherwise noted, all parts, percentages, ratios, etc. in the Examples and the rest of the specification are by weight. These abbreviations are used in the following examples: g=grams, min=minutes, hr=hour, mL=milliliter, L=liter. If not otherwise indicated in the table, below, chemicals were obtained from Sigma-Aldrich, St. Louis, Mo.

Materials utilized for the examples are shown in Table 1.

TABLE 1
Materials List

| Material | Description | Source |
|---|---|---|
| VCl₃ | Vanadium chloride, CAS No.7718-98-1 | Alfa Aesar, |
| | | Ward Hill, MA |
| C₁₂H₁₂N₂O₂S | Dapsone, CAS No. 80-08-0 | Sigma-Aldrich, |
| | | St. Louis, MO |
| Tsuda's reagent | N-(2-Diethylaminoethyl)-1-naph- | TCI America, |
| | thylamine oxalate, CAS 29473-53-8 | Portland, OR |
| 1N HCl | Hydrochloric acid, CAS 7647-01-1 | VWR, Radnor, |
| | | PA |
| KNO₃ | Potassuim Nitrate, CAS No. 7757-79-1 | EMD Science, |
| | | Darmstadt, |
| | | Germany |
| ZnSO₄•7H20 | Zinc sulfate heptahydrate, CAS | EMD Science, |
| | No. 7446-20-0 | Darmstadt, |
| | | Germany |
| Saline | 0.9% sodium chloride injection, USP | Baxter, |
| | | Deerfield, IL |

### Sample Acquisition

A nitrate-free absorbent pad was used to collect wound fluid, namely a NEXCARE Soft 'n Flex first aid dressing (catalog no. 672-35), available from 3M Company (St. Paul, Minn.). Examples of such pads are described in International Publication No. WO 2011/017325. The pad was placed in contact with the wound for sufficient time to absorb at least 10 μL fluid, typically less than 1 minute. After collection, the pad was placed in a polypropylene tube and frozen at −80° C. until analysis.

### Reagent Solutions

A 1.5% ZnSO₄ solution was prepared by dissolving approximately 0.268 g ZnSO₄.7H₂O in deionized water.

A 0.8% VCl₃ reagent mix was prepared by dissolving 0.45 g VCl₃ in 1N HCl in a brown glass bottle, working in an inert environment (nitrogen-purged glove bag). Dapsone (0.14 g) and Tsuda's reagent (0.02835 g) were added to the VCl₃ and the bottle shaken until dissolved.

### Sample Analysis

The collected samples were analyzed as follows:
1. Thaw the frozen samples to room temperature and transfer the sample acquisition device to a pre-washed test tube containing a frit
2. Centrifuge the test tube to separate the wound fluid from sample acquisition device
3. Remove protein from the samples
   a. Transfer 10 μL wound fluid to a pre-washed test tube
   b. Add 10 μL 1.5% ZnSO₄ solution
   c. Vortex
   d. Add 70 μL water
   e. Vortex, centrifuge 5 min at 10,000 rpm
   f. Transfer 75 μL of supernatant to new pre-washed polypropylene tube
4. Add 10 μL VCl₃ reagent mix to the sample
5. Vortex, centrifuge 2 minutes at 5000 rpm
6. Heat at 70° C. with agitation for 10 minutes with an Eppendorf Thermomixer R Dry Block Heating and Cooling Shaker (Eppendorf North America, New York, N.Y.)
7. Let cool 2-3 min
8. Vortex
9. Centrifuge for 2 minutes at 5000 rpm
10. Transfer 75 μL of the liquid to a 384 well plate
11. Measure the absorbance of the sample at 545 nm and at 450 nm with a BioTek Synergy H4 Hybrid Multi-Mode Microplate Reader (BioTek US, Winooski, Vt.)
12. Subtract the absorbance at 450 nm from the absorbance at 545 nm. This absorbance value is used with the standard curve to calculate the concentration of nitrate in the sample

### Standard Curve

Potassium nitrate was used to prepare a series of nitrate standards in saline over the expected nitrate range in the samples. These standards were analyzed per the method described in Sample Analysis, starting with Step 3. This assay is linear up to at least 200 μM.

### Samples 1-16

Fluid from the wound of 16 different patients was collected and analyzed per the method described. Each sample was assayed in triplicate. Table 2 contains the individual replicates as well as the mean values, standard deviations, and coefficients of variation. These data demonstrate the reproducibility of the developed assay over a broad range of NOx values.

TABLE 2
NOₓ Levels in Patients

| Sample | | | | Mean | Std | CV |
|---|---|---|---|---|---|---|
| ID | Replicate values | | | [NOx], μM | Dev | (%) |
| 1 | 66.82 | 75.00 | 75.91 | 72.6 | 2.0 | 2.75 |
| 2 | 44.14 | 45.10 | 53.67 | 47.6 | 5.2 | 10.92 |
| 3 | 27.48 | 27.48 | 26.05 | 27.0 | 0.8 | 2.96 |
| 4 | 14.62 | 10.81 | 8.90 | 11.4 | 2.9 | 25.44 |
| 5 | 25.38 | 30.14 | 28.24 | 27.9 | 2.4 | 8.60 |
| 6 | 117.14 | 133.95 | 138.95 | 130.0 | 11.4 | 8.77 |
| 7 | 26.23 | 31.23 | 29.86 | 29.1 | 2.6 | 8.93 |
| 8 | 65.32 | 74.86 | 74.41 | 71.5 | 5.4 | 7.55 |
| 9 | 47.19 | 43.38 | 46.24 | 45.6 | 2.0 | 4.35 |
| 10 | 19.57 | 21.48 | 21.00 | 20.7 | 1.0 | 4.79 |
| 11 | 31.10 | 33.60 | 32.60 | 32.4 | 1.3 | 3.88 |
| 12 | 23.60 | 28.60 | 25.10 | 25.8 | 2.6 | 9.96 |
| 13 | 15.60 | 16.60 | 16.10 | 16.1 | 0.5 | 3.11 |
| 14 | 16.10 | 15.60 | 17.10 | 16.3 | 0.8 | 4.70 |
| 15 | 20.10 | 18.10 | 17.60 | 18.6 | 1.3 | 7.11 |
| 16 | 129.60 | 124.10 | 139.10 | 130.9 | 7.6 | 5.80 |

### Clinical Study

A clinical study was performed to assess the relationship between wound healing and NOx levels in wound fluid. Three levels (I, II, and III) of WFNOx activity (μM) were identified. The two clinical assessments were: 1) wound progression (P)—with decreasing wound area, increased granulation tissue deposition and wound closure; or, 2) wound non-progression and/or worsening (NP/W)—with increasing wound areas, wound bed stagnation and/or excessive wound inflammation.
Level I (NP/W)—WFNOx at or below 10 or 15 μM; wound non-progression
Level II (P)—WFNOx from 10 or 15 μM to 50 or 60 μM; wound progression
Level III (NP/W)—WFNOx at or above 50 or 60 μM; wound non-progression

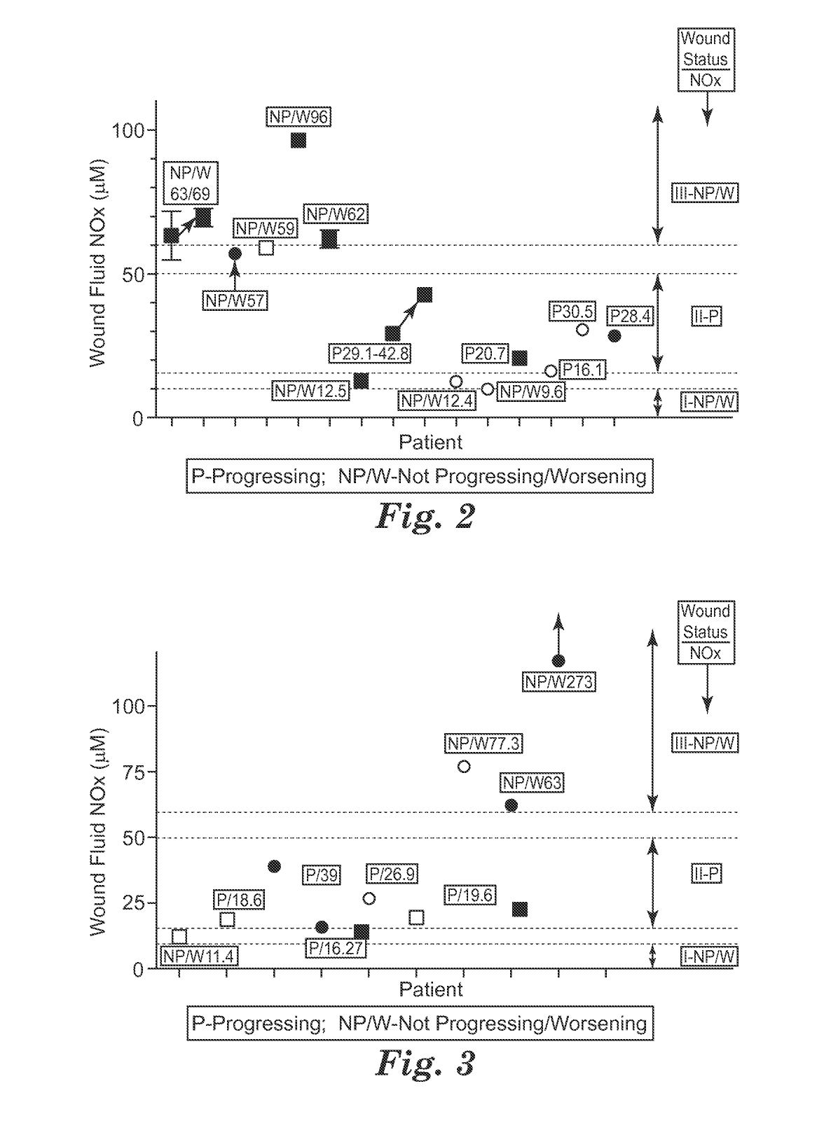

Data from venous leg ulcer (VLU) patients are show in FIG. 2 and from diabetic foot ulcer (DFU) patients in FIG. 3. The analytical method described above was utilized to measure the NOx levels.

### Patient Treatment

Based on the NOₓ concentrations in the patient wound fluid, particular treatments may be proposed. For elevated concentrations, the use of glucocorticoids topically or prednisone therapy may be utilized to bring the concentration back to the therapeutic range. Patients with depressed levels of NOx in wound fluid may be assessed for nutritional status and consideration may be given to supply arginine supplements. ARGINAID (Nestle, Highland Park, Mich.), arginine tablets, or gelatin may be provided to the patients to increase NOx levels.

The complete disclosures of the patents, patent documents, and publications cited herein are incorporated by reference in their entirety as if each were individually incorporated. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

1.-35. (canceled)

36. A method of promoting wound healing in a mammal, the method comprising:
treating the mammal with a substance that alters the wound fluid nitric oxide (WFNO) level such that a therapeutic window of WFNO in the wound is established thereby promoting wound healing in the mammal;
wherein the therapeutic window of WFNO is established by a method comprising
obtaining a wound fluid sample from a mammal;
analyzing the WFNO level;
determining whether the WFNO is at or below a lower threshold level, or is at or above an upper threshold level; wherein the lower threshold level and upper threshold level define the therapeutic Window of WFNO.

37. The method of claim 36 wherein analyzing the WFNO level comprises analyzing the amount of nitric oxide, a nitrate, a nitrite, or a combination thereof, in the wound fluid.

38. The method of claim 37 wherein analyzing the WFNO level comprises analyzing the amount of a nitrate, a nitrite, or a combination thereof, in the wound fluid.

39. The method of claim 38 wherein analyzing the WFNO level comprises measuring the amount of NOx in the wound fluid.

40. The method of claim 39 wherein the lower threshold level of NOx is no greater than 10 micromolar.

41. The method of claim 39 wherein the upper threshold level of NOx is no less than 60 micromolar.

42. The method of claim 36 wherein obtaining a wound fluid sample from a mammal comprises:
providing a sample acquisition device that is substantially free of reactive nitrates;
contacting the sample acquisition device with a wound site for a period of time sufficient to collect wound fluid; and
extracting a portion of the wound fluid from the sample acquisition device.

43. The method of claim 36 wherein analyzing the WFNO level comprises:
providing a reducing agent capable of reducing a nitrate compound to a nitrite compound;
providing a chromogenic reagent capable of reacting with a nitrite compound to form a colored compound;
contacting the chromogenic reagent and reducing agent with the wound fluid sample under conditions that permit the reduction of a nitrate compound to a nitrite compound and permit the reaction of a nitrite compound with the chromogenic reagent to form the colored compound; and
detecting the colored compound.

44. The method of claim 43 wherein analyzing the WFNO level comprises:
providing a first retention medium configured to retain the colored compound forming a first mixture comprising the sample, the reducing agent, and the chromogenic reagent under conditions that permit the reduction of a nitrate compound to a nitrite compound and permit the reaction of a nitrite compound with the chromogenic reagent to form the colored compound;
contacting at least a portion of the first mixture with the first retention medium; and
detecting the colored compound retained on the first retention medium.

45. The method of claim 36 wherein the step of treating the mammal with a substance that alters the wound fluid nitric oxide comprises increasing the WFNO level in the wound.

46. The method of claim 45 wherein the step of treating comprises administering L-arginine to the mammal at a therapeutically effective dose, performing hyperbaric oxygen therapy on the mammal, treating the wound with topical nitric oxide in a gaseous form, applying a nitric oxide donor to the wound, applying a nanocrystalline NO powder or nitroglycerin paste to the wound, or combinations thereof.

47. The method of claim 36 wherein the step of treating the mammal with a substance that alters the wound fluid nitric oxide comprises decreasing the WFNO level in the wound.

48. The method of claim 47 wherein the step of treating comprises administering a nitric oxide inhibitor, an inhibitor of nitric oxide synthase, herbs with nitric oxide scavenging activity, a steroid, or applying a topical corticosteroid preparation, or a combination thereof.

49. A method of establishing a therapeutic window of wound fluid nitric oxide (WFNO) in the wound of a mammal, the method comprising:
obtaining a wound fluid sample from a mammal;
analyzing the WFNO level; wherein WFNO comprises a nitrate, a nitrite, or a combination thereof, in the wound fluid;
determining whether the WFNO is at or below a lower threshold level, or is at or above an upper threshold level; wherein the lower threshold level and upper threshold level define the therapeutic window of WFNO; and
treating the mammal with a substance that alters the WFNO level such that the therapeutic window of WFNO in the wound is established thereby allowing for improved wound healing.

50. The method of claim 49 wherein obtaining a wound fluid sample from a mammal comprises:
providing a sample acquisition device that is substantially free of reactive nitrates;
contacting the sample acquisition device with a wound site for a period of time sufficient to collect wound fluid; and
extracting a portion of the wound fluid from the sample acquisition device.

51. The method of claim 50 wherein analyzing the WFNO level comprises:
providing a reducing agent capable of reducing a nitrate compound to a nitrite compound;
providing a chromogenic reagent capable of reacting with a nitrite compound to form a colored compound;
contacting the chromogenic reagent and reducing agent with the wound fluid sample under conditions that permit the reduction of a nitrate compound to a nitrite compound and permit the reaction of a nitrite compound with the chromogenic reagent to form the colored compound; and
detecting the colored compound.

52. The method of claim 51 wherein analyzing the WFNO level comprises:
providing a first retention medium configured to retain the colored compound forming a first mixture comprising the sample, the reducing agent, and the chromogenic reagent under conditions that permit the reduction of a nitrate compound to a nitrite compound and permit the reaction of a nitrite compound with the chromogenic reagent to form the colored compound;
contacting at least a portion of the first mixture with the first retention medium; and
detecting the colored compound retained on the first retention medium.

53. The method of claim 49 wherein the step of treating the mammal to establish a therapeutic window of WFNO in the wound comprises increasing the WFNO level in the wound.

54. The method of claim 49 wherein the step of treating the mammal to establish a therapeutic window of WFNO in the wound comprises decreasing the WFNO level in the wound.

55. A method of establishing a therapeutic window of wound fluid NO (WFNO) in the wound of a mammal, the method comprising:
obtaining a wound fluid sample from a mammal; wherein obtaining a wound fluid sample comprises:
providing a sample acquisition device that is substantially free of reactive nitrates;
contacting the sample acquisition device with a wound site for a period of time sufficient to collect wound fluid; and
extracting a portion of the wound fluid from the sample acquisition device;
analyzing the WFNO level; wherein WFNO comprises a nitrate, a nitrite, or a combination thereof, in the wound fluid; and further wherein analyzing the WFNO level comprises:
providing a reducing agent capable of reducing a nitrate compound to a nitrite compound;
providing a chromogenic reagent capable of reacting with a nitrite compound to form a colored compound;
contacting the chromogenic reagent and reducing agent with the wound fluid sample under conditions that permit the reduction of a nitrate compound to a nitrite compound and permit the reaction of a nitrite compound with the chromogenic reagent to form the colored compound; and
detecting the colored compound;
determining whether the WFNO is at or below a lower threshold level, or is at or above an upper threshold level; wherein the lower threshold level and upper threshold level define the therapeutic window of WFNO; and
treating the mammal with a substance that alters the WFNO level such that after said treating, the therapeutic window of WFNO in the wound is established thereby allowing for improved wound healing.

56. An in vitro method of monitoring the effectiveness of the treatment of a wound in a mammal, the method comprising:
analyzing the level of wound fluid nitric oxide (WFNO) in a wound fluid sample obtained from the wound of the mammal; and
determining whether or not the WFNO level in the wound is between a lower threshold level and an upper threshold level required for wound healing.

57. The method of claim 56 wherein the lower threshold level required for wound healing is 10 micromolar.

58. The method of claim 56 wherein the upper threshold level required for wound healing is 60 micromolar.
